(19) United States
(12) Patent Application Publication
(10) Pub. No.: US 2025/0288206 A1
(43) Pub. Date: Sep. 18, 2025

(54) SYSTEMS AND METHODS FOR MONITORING OF DELIVERY OF LABEL-FREE DRUGS

(71) Applicant: City University of Hong Kong, Hong Kong (HK)

(72) Inventors: Wai Yan Kannie CHAN, Hong Kong (HK); Jianpan HUANG, Hong Kong (HK); Haoyun SU, Hong Kong (HK)

(73) Assignee: City University of Hong Kong, Hong Kong (HK)

(21) Appl. No.: 18/607,920

(22) Filed: Mar. 18, 2024

(51) Int. Cl. A61B 5/00; A61B 5/055; A61K 49/18

(52) U.S. Cl. CPC A61B 5/0036; A61B 5/055; A61K 49/1812

(57) ABSTRACT

There is disclosed a system for delivery of a drug in vivo. In the delivery system, the extent of the delivery of the drug is monitorable and/or controllable by using chemical exchange saturation transfer magnetic resonance imaging (CEST MRI). The delivery system has elements of a solvent system of dimethyl sulfoxide (DMSO) or its analogs for carrying the drug, detecting the delivery and/or biodistribution the drug, and/or interaction of the drug compartmentalized in vivo by way of directly imaging the solvent system by the CEST MRI due to the inherent contrast of the solvent system.

### FIELD OF THE INVENTION

The present invention is concerned with a label-free and non-invasive system and method to image or otherwise monitor the delivery of drugs in vivo, and in particular but not limited to using chemical exchange saturation transfer magnetic resonance imaging (CEST MRI) for imaging, monitoring and/or controlling the delivery a drug.

### BACKGROUND OF THE INVENTION

Monitoring of the delivery of a drug in vivo is important tool because it can provide a picture on the location, extent and/or biodistribution of the drug concerned. In the past, there have been various conventional approaches. However, many of these approaches involve the labeling of the drug concerned, and/or the use of metallic or fluorescence labels, or the use of invasive procedures. These approaches are far from ideal for many reasons. For example, the labels themselves may affect the efficacy of the drug or may elicit adverse reactions from the patient concerned.

In view of the aforementioned problems, the present invention seeks to overcome these problems or at least to provide the public with an alternative.

### BRIEF DESCRIPTION OF THE DRAWINGS

Some embodiments of the present invention will now be explained, with reference to the accompanied drawings, in which:

FIG. 1A is a schematic diagram showing a contrast mechanism of DMSO CEST signal, and FIGS. 1B to 1C, FIGS. 1D to 1E and FIGS. 1F to 1G are graphs showing characteristic Z-spectra and trendlines for i) concentration, ii) pH and iii) temperature dependence of DMSO CEST, respectively.

FIGS. 2A to 2B and FIGS. 2C to 2D are graphs illustrating the effect of B₁ and Tₛₐₜ on saturation dependence of DMSO CEST contrast, respectively.

FIGS. 3A to 3C illustrate aprotic analogs of DMSO demonstrated discernable CEST peaks at the negative offset range, in which FIG. 3A is a graph showing the characteristic Z-spectra of DMSO, acetone and acetonitrile, FIG. 3B and FIG. 3C are graphs showing i) dependence of aprotic analogs' CEST peak magnitude on B₁ and concentration, respectively.

FIGS. 4A to 4C are graphs illustrating protic analogs of DMSO demonstrated discernable CEST peaks at the negative offset range, in which FIG. 4A shows the characteristic Z-spectra of alcohols at 0.6 μT, and FIG. 4B and FIG. 4C show the dependence of protic analogs' CEST peak magnitude on B₁ and concentration, respectively.

FIGS. 5A to 5C are graphs illustrating CEST measurement and release study of BA-DMSO liposome (n=3), in which FIG. 5A shows the CEST peaks of BA-DMSO liposome before dialysis, 0.25, 0.5, 1 and 3 hour(s) after dialysis, FIG. 5B shows the normalized CEST intensity of BA and DMSO at different timepoints, and FIG. 5C shows the cumulative drug release of BA.

FIG. 6 is a graph illustrating the Bloch-McConnell fitted CEST peak for DMSO.

FIGS. 7A to 7B, 7C to 7D and 7E to 7F are graphs illustrating the averaged Z-spectra and MTR_{asym} with error bars for i) DMSO, ii) acetone and iii) acetonitrile aprotic solvents at 10% concentration and Tₛₐₜ=4 s (n=3), respectively.

FIGS. 8A to 8B, 8C to 8D, 8E to 8F and 8G to 8H are graphs illustrating the averaged Z-spectra and MTR_{asym} with error bars for i) methanol, ii) ethanol, iii) 1-Propanol, and iv) 2-Propanol protic solvents at 10% concentration and Tₛₐₜ=4 s (n=3), respectively.

### SUMMARY OF THE INVENTION

According to a first aspect of the present invention, there is provided a system for delivery of a drug in vivo wherein the extent of the delivery of the drug is monitorable and/or controllable by using chemical exchange saturation transfer (CEST) magnetic resonance imaging (MRI), comprising a solvent system of dimethyl sulfoxide (DMSO) or its analogs for carrying the drug, detecting the delivery and/or biodistribution of the drug, and/or interaction of the drug compartmentalized in vivo by way of directly imaging the solvent system by the CEST MRI due to the inherent contrast of the solvent system.

Preferably, the drug may be a hydrophobic drug, an anticancer drug, a small molecule drug, or a macromolecular drug selected from a group including a DNA, RNA, or peptide.

Suitably, the drug may be free of any label.

Advantageously, the solvent system or the drug carried by the solvent system may be free of any metallic, radioactive or fluorescent dye or label.

The workable concentration of the drug in the solvent system (e.g. DMSO or an analog thereof) would depend on types of drugs, clinical guidelines on dosing, and solubility property of the drug itself. It can range from UM to mM concentration. In the context of the present invention, and with respect to the drug barbituric acid (BA), the concentration of 100 mM BA in DMSO system or aqueous system without DMSO can be used, as supported by experiments. This concentration translates to about 12.81 mg/ml. However, further experiments show that the delivery can support a broad range of concentration. For example, multiple studies show that the solvent system can support a relative wide range of concentration of the drug in the solvent system. Studies leading to the present invention has shown that when the drug is a hydrophobic drug the concentration of the hydrophobic drug in the solvent system may preferably at 1 mM to 45 mM, and when the drug is a macromolecular drug the concentration of the macromolecular drug may preferably at 0.1 μg/μL to 2 μg/μL.

In an embodiment, the concentration of the DMSO or its analogs in the solvent may be 1.25 to 20% v/v.

In one embodiment, CEST effects of the DMSO or its analogs may range from −1.4 ppm to −3.8 ppm.

The analogs of the solvent system may be selected from a group including aprotic solvents and protic solvents.

The analogs of the solvent system may be selected from the group consisting of acetone, acetonitrile, methanol, ethanol, 1-propanol and 2-propanol.

The aprotic and protic solvents of the analogs may be both polar and having methyl groups therein.

The delivery may comprise a nanoparticle carrier for the drug, wherein the nanoparticle carrier may be liposome. The solvent system loaded with drug may then co-loaded with the liposome. The liposome may be used as a probe for monitoring of delivery of the drug.

The monitoring system may further comprise a MRI-trackable nanocarrier of the drug in the solvent system. The MRI-trackable nanocarrier may be adapted to be controllable for release of the drug when the drug reaches certain location or circumstances in vivo.

According to a second aspect of the present invention, there is provided a method of using chemical exchange saturation transfer (CEST) magnetic resonance imaging (MRI) for monitoring delivery and/or controlling a release of a drug in vivo, comprising the steps of: i) providing a drug to be delivered, ii) providing a solvent system of dimethyl sulfoxide (DMSO) or its analogs for carrying the drug, and iii) using CEST MRI to detect the delivery and/or biodistribution the drug, and/or interaction of the drug compartmentalized in vivo by way of directly imaging the solvent system by the CEST MRI due to the inherent contrast of the solvent system.

Preferably, the drug may be selected from a group selected from a hydrophobic drug, an anticancer drug, a small molecule drug, a peptide and a gene, and the drug may be free of any label, and/or the solvent or the drug may be free of any metallic, radioactive or fluorescent dye or label.

Suitably, when the drug is a hydrophobic drug the concentration of the hydrophobic drug in the solvent system may be at 1 mM to 45 mM, or when the drug is a macromolecular drug the concentration of the macromolecular drug is at 0.1 μg/μL to 2 μg/μL the concentration of the DMSO or its analogs in the solvent may be 1.25 to 20% v/v.

Advantageously, the solvent of analogs may be selected from the group including aprotic solvents and protic solvents, or is acetone, acetonitrile, methanol, ethanol, 1-propanol or 2-propanol.

In an embodiment, the method may comprise a nanoparticle carrier for the drug, wherein the nanoparticle carrier may be liposome, and wherein the solvent system loaded with the drug may then be co-loaded with the liposome, wherein the liposome may be used as a probe for monitoring of delivery of the drug.

In one embodiment, the method may comprise a MRI-trackable nanocarrier of the drug in the solvent system, wherein the MRI-trackable nanocarrier may be adapted to be controllable for release of the drug when the drug reaches certain location or circumstances in vivo.

### DETAILED DESCRIPTION OF THE INVENTION

Dimethyl sulfoxide (DMSO) has wide biomedical applications such as cryoprotectant and hydrophobic drug carrier. The present invention as described below demonstrate that DMSO can generate a distinctive CEST peak at around −2 ppm. Structural analogs of DMSO sharing the common characteristics, including aprotic and protic solvents, also demonstrated CEST signals from −1.4 ppm to −3.8 ppm. The present invention illustrates that the intermolecular interaction between DMSO and water creates a solvation shell (“structured” water) that can serve as relaying sites where RF-labeled aliphatic protons can transfer saturation to the “structured” water protons. Subsequently, the saturation is transferred from “structured” water protons to bulk water protons via chemical exchange, resulting in the observed peaks at negative offset frequencies. When CEST detectable barbituric acid (BA) is dissolved in DMSO solution and are co-loaded to liposome, two obvious peaks at 5 ppm and −2 ppm are observed. DMSO's CEST effect can be attributed to the hydration shell around DMSO molecule. This locally structured water around solvent molecule that originates from the intermolecular interaction between DMSO and nearby water molecules, can be the source of the observed DMSO CEST contrast. The findings from the present invention indicate that such solvent-water interaction is a key factor in generating CEST effect at negative offset frequencies.

Dimethyl sulfoxide (DMSO) is a versatile compound that has been used as a reagent and solvent in organic synthesis and industrial processes, as a cryoprotectant in cultured mammalian cells, as a carrier for hydrophobic drugs, and as a US Food and Drug Administration (FDA) approved active pharmaceutical ingredient for treating interstitial cystitis. This unique solvent system has attracted attention, especially for studying the interaction between DMSO and water at a molecular level to facilitate the delivery of hydrophobic drugs. Spectroscopic and simulation studies showed that intermolecular interaction, such as hydrogen bonding (H-bonding), exists between DMSO and nearby water. When molar fraction of DMSO is less than 10% in DMSO-H₂O mixture, DMSO molecules are solvated in the form of 1DMSO-2H₂O complexes, as observed by multiple studies. Thus, probing and characterizing the molecular interaction was crucial to DMSO's biomedical application.

Chemical exchange saturation transfer (CEST) MRI detects exchangeable protons of many endogenous and exogenous molecules. This exchange between the solute pool and water pool amplifies the signal, which enables the detection of proton exchange related events in a sensitive manner in vivo, including pH, temperature and H-bonding. On the downfield side at 1-4 ppm, i.e. positive offset frequency from water signal, amide proton transfer (APT), creatine, glutamate and glucose are extensively studied. Molecules with intramolecular H-bonding have shifted the CEST effect further down to 7-9 ppm. At the negative offset frequency (from around −1 to −5 ppm), relayed Nuclear Overhauser Effect (rNOE), where non-exchangeable aliphatic protons transfer magnetization to nearby dipolar-coupled protons and subsequently exchange with bulk water, is observed. While both APT and rNOE have been applied to study brain tumors for tumor grading and assessment of therapeutic outcomes, CEST effect of other molecules, such as glycogen, lipid, and bound small molecules such as lactate and caffeine are also known to have rNOE signals. Given its unique contrast mechanism, CEST MRI is well-suited for detecting molecular interactions.

Liposome is a well-known nano-sized drug delivery vehicle for a wide range of applications, including anticancer, antibacterial, and antiviral therapies. Since the first liposomal formulation for cancer therapy, Doxil®, was approved by the US FDA in 1995, there has been an increasing number of FDA-approved liposome-based therapies with encapsulated agents such as small molecular drugs. During the course leading the present invention, it has been demonstrated that, in the context of the present invention, characteristics of liposome, such as the permeable lipid bilayer and preferably high surface-to-volume ratio, have made it an ideal CEST probe where encapsulated CEST agents can be detected with increased sensitivity. Moreover, the phospholipid bilayer can be detected by CEST MRI at −3.5 ppm as well.

The present invention illustrates that DMSO-water mixture can generate CEST contrast at a negative offset frequency for the first time. CEST properties of DMSO as well as those of DMSO's structural analogs were measured. The present invention also demonstrates the potential application of DMSO solvent system in liposomal drug delivery, which can be applied as a theranostic agent.

### Material and Methods

DMSO, acetone, acetonitrile, methanol, ethanol, 1-propanol, and 2-propanol (isopropanol) with purity ≥99% were purchased from Sigma-Aldrich. Cholesterol (Chol), and barbituric acid (BA) were purchased from Sigma-Aldrich. HEPES were purchased from Supelco. DSPC and DSPE-PEG2000 were purchased from Avanti Polar Lipids.

### Phantom Solution and Liposome Preparation

Phantom solutions were prepared by mixing 10% v/v DMSO or analogs in 20 mM HEPES buffered saline (HBS), and were adjusted to various pH (5.5-8, 0.5 increment) then topped up to final 10% concentration. For concentration dependence measurement, solvent samples were prepared at concentration of 20, 10, 5, 2.5, and 1.25% v/v. A list of analog solvents used in this study was shown in below Table 1.

TABLE 1
List of solvents used in CEST experiment and their respective observed CEST effect offsets.

| | | | Observed CEST |
|---|---|---|---|
| Solvents | | | Contrast Offset (ppm) |
| Polar aprotic | DMSO | 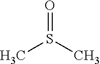 | −2 |
| | Acetone | 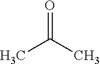 | −2.5 |
| | Acetonitrile | 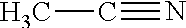 | −2.5 |
| Polar protic | Methanol | H₃C—OH | −1.4 |
| | Ethanol | 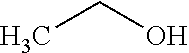 | −3.6 |
| | 1-Propanol | 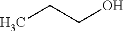 | −3.8 |
| | 2-Propan ol (Isopropanol) | 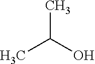 | −3.6 |

100 mM BA in 10% v/v DMSO solution at pH 7.2 were prepared in the same manner as phantom solutions and were used in the thin-film rehydration method of liposome preparation. Briefly, DSPC, Chol, and 18:0 PEG2000 PE dissolved in chloroform were mixed at a molar ratio of 50:40:10 (25 mg of total lipids). The mixture was dried using a rotary evaporator (Heidolph, Schwabach, Germany) for 1.5 hours at 75 rpm, to remove residual chloroform and form a lipid thin film. Subsequently, 2 mL of 10% BA-DMSO solution was used to rehydrate the lipid film at 55° C. for 1 hour with intermittent agitation to obtain large vesicle solution. Then, a bath type sonicator was used to sonicate the vesicle solution for 30 minutes. Liposome solution was then extruded with 400 nm polycarbonate filters (Whatman, Maidstone, UK), and subsequently with 200 nm filters. Unencapsulated BA was removed by passing liposome solution through Sephadex G50 gel columns (GE Healthcare, Chicago, Illinois, USA). The final BA-DMSO liposome solution was filtered through 0.2 μm syringe filters and kept in a 4° C. refrigerator for future use. All the solution and liposome experiments were carried out independently in triplicates.

### Characterization of Physicochemical Properties and Release Profile of BA-DMSO Liposome

The size, polydispersity index (PDI), and surface charge of DMSO liposome were measured by dynamic light scattering using Zetasizer (Malvern Instruments, UK). Liposome concentration was measured by light scattering using NanoSight (Malvern Instruments, UK). To study the release property, dialysis cassettes with molecular weight cut-off of 10 kDa (Thermo Fisher, Waltham, USA) were used. The cassettes were first hydrated in HBS for 2 minutes. 1 mL of liposome sample was then loaded into the membrane chamber of two separate cassettes and excess air was removed by gentle squeezing of the membrane. The loaded cassettes were submerged into two beakers with 50 mL HBS at 37° C. At 15 min, 30 min, 1 hour, and 3 hour, 200 μL of dialyzed liposomes were taken out for CEST MRI measurement. In the other dialysis cassette, 1 mL of dialysis medium were taken out from the cassette at each timepoint for UV spectrometric measurement at an absorbance of 257 nm, and 1 mL of fresh medium was added after each time the medium was taken out. The retention of drug within liposome before and after dialysis was determined by measuring the UV absorbance of the supernatant obtained by treating liposome solutions with 10% Triton-X solution, sonicating at 42° C. for 1 hour, and centrifuging at 10,000 rpm. Concentration of BA was determined from BA standard curve.

### CEST Imaging of Phantoms

All imaging sessions were done using Bruker Biospec 3T (Bruker, Ettlingen, Germany), with a 40-mm volume transceiver coil. 200 μL of solution or liposome sample were loaded to 6 mm glass NMR tube for MRI acquisition. The tubes were placed parallel to the magnetic field. B₀ was shimmed to first-order using water linewidth. A modified RARE sequence with a continuous wave (CW) saturation module was used to acquire Z-spectrum. Specific parameters were: slice thickness=2 mm, field of view (FOV)=30×30 mm², image size=64×64, RARE factor=32, TR=9000 ms, TE=75 ms. For power dependence experiment, Z-spectra were acquired at B₁ power of 0.4, 0.8, 1.2, 1.5 and 2.0 μT, with a saturation duration (Tₛₐₜ) of 4 s. For saturation time dependence experiment, Z-spectra were acquired at Tₛₐₜ of 1, 2, 3, 4, 5, and 6 s, with a B₁ power of 1.2 μT. The saturation frequency offset ranged from −10 to 10 ppm. An offset step size of 0.1 ppm was used between 2.1 and −2.1 ppm, and a step size of 0.2 ppm was used for the remainder of offset range. For the analogs, a step size of 0.1 ppm was used in ±0.3 ppm of their respective offset frequency, a step size of 0.05 ppm was used from −0.5 ppm to 0.5 ppm, and a step size of 0.2 ppm was used for the rest of the offset frequencies. Z-spectrum was processed with direct water saturation removal by Lorentzian fitting with our custom Matlab codes (Mathworks, Natick, USA).

### Results

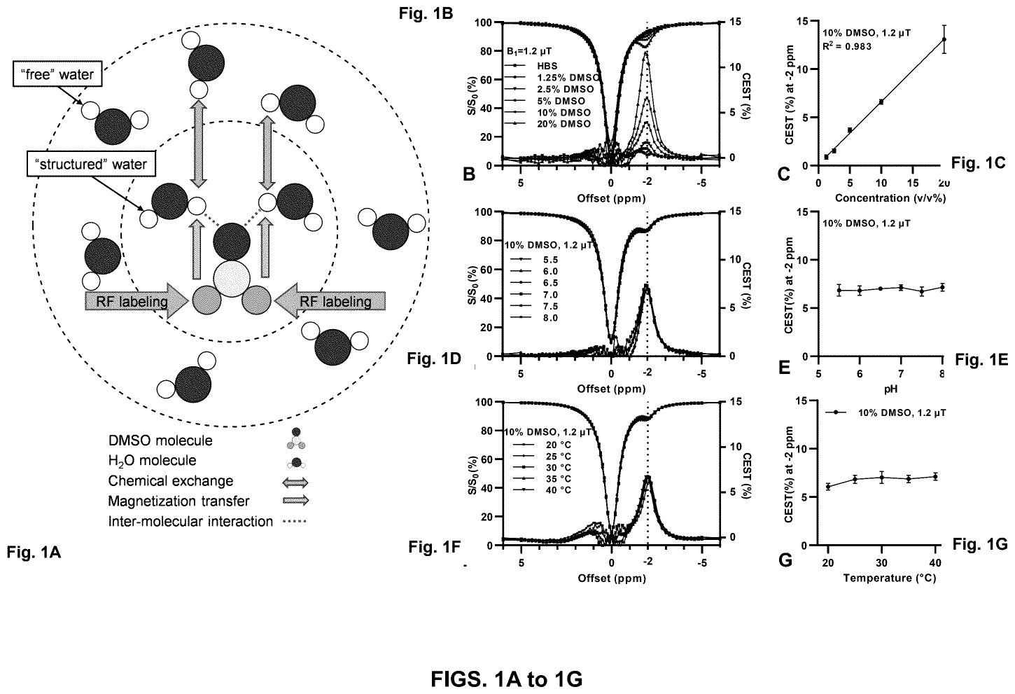

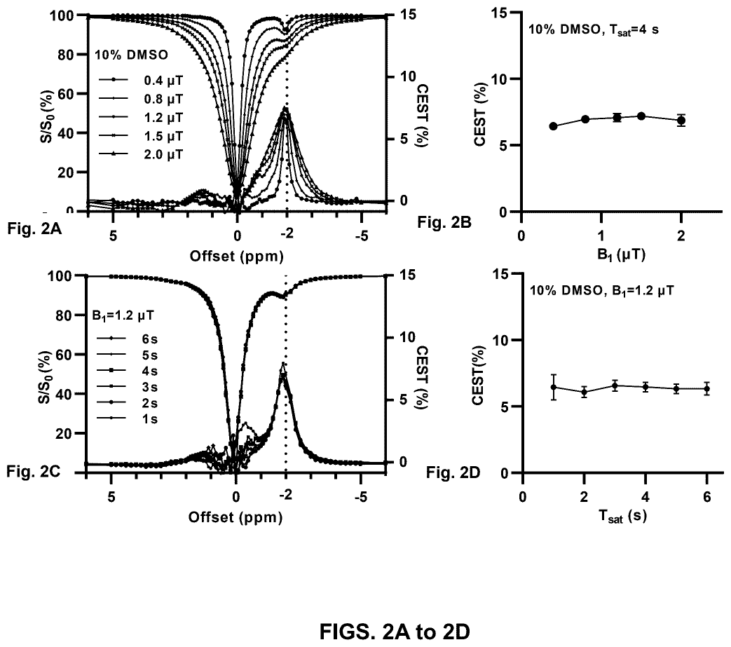

DMSO mixed with HBS from 1.25 to 20% v/v showed a distinctive peak at around −2 ppm. External factors were altered to study the characteristics of DMSO CEST peak. The signal magnitude of DMSO increased with the increasing concentration at 1.2 μT, while pH (5.5-8.0) effect on the signal was less prominent. Please see FIGS. 1B to 1E. However, as the temperature increased from 20° C. to 40° C., signal offset went slightly closer to water peak. For B₁ power and Tₛₐₜ, dependence, DMSO signal slightly increased when power was increased from 0.4 to 2.0 μT, and stayed relatively stable when saturation duration was increased from 1 s to 6 s. Please see FIGS. 2A to 2D.

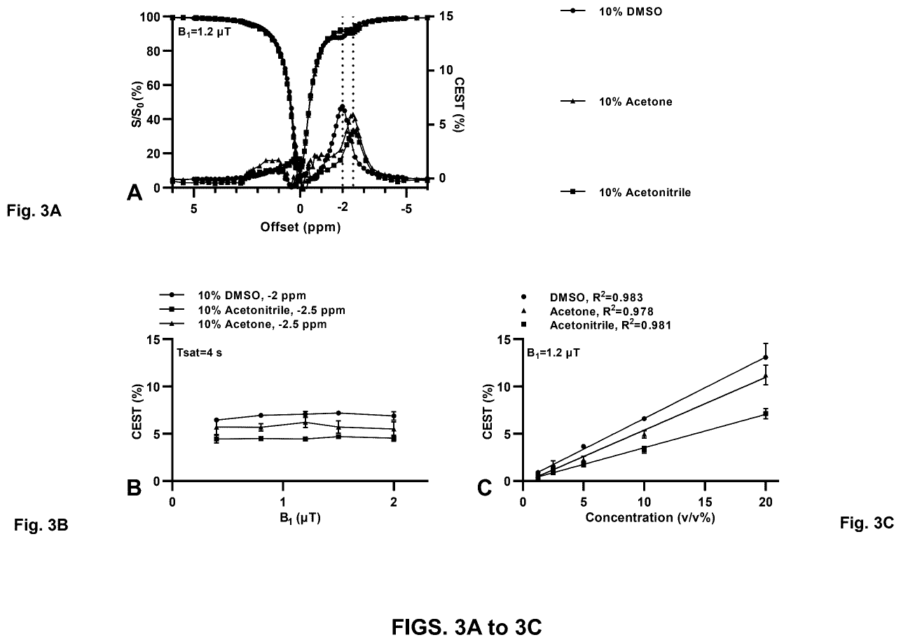

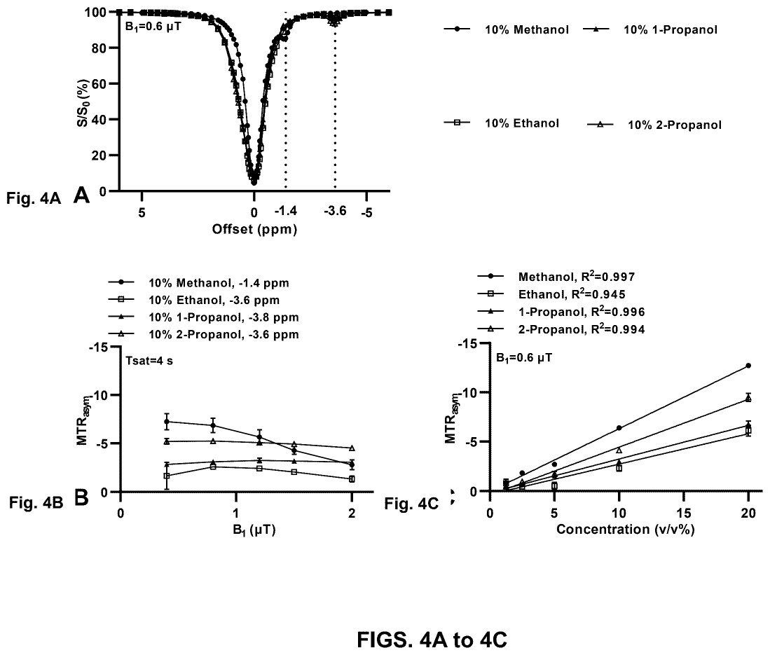

To further demonstrate the underlying contrast mechanism of DMSO, a series of solvents that are structural analogs to DMSO has been identified. Acetone and acetonitrile which are both polar and having methyl groups in their structures, were selected as aprotic solvent analogs to DMSO. Methanol, ethanol, 1-propanol and 2-propanol were selected as protic solvent analogs to DMSO. A list of the solvents and their respective observed offsets are shown in above Table 1. Results have demonstrated that all these analogs can generate discernable CEST peaks at the negative offset range. Please see FIGS. 3A to 3B and FIGS. 4A to 4B.

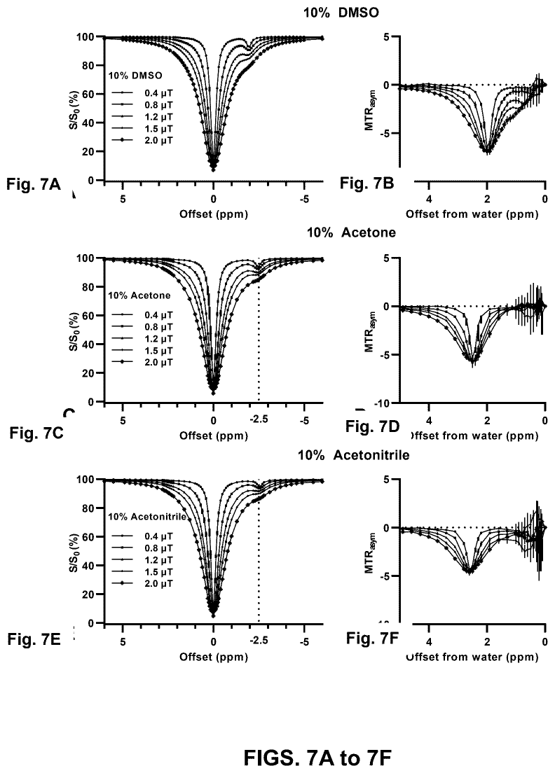

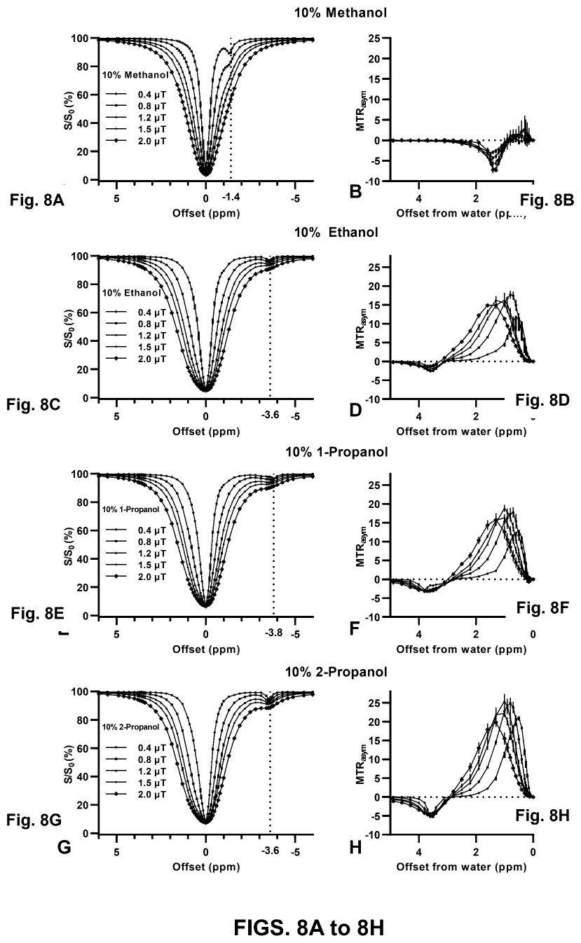

Compared within aprotic group, acetone and acetonitrile's peaks were further upfield at around −2.5 ppm, and were less in magnitude compared to DMSO's peak. Please see FIGS. 3A to 3B. The CEST peaks of three aprotic analogs shared similar trend when varying B₁, and also scaled with concentration. For the four alcohols, they also had CEST effects from −1.4 ppm to −3.8 ppm. Characteristic Z-spectra of alcohols at 0.6 μT were shown in the figure in order to demonstrate methanol's CEST effect which was more prominent at low power. Please see FIG. 4B. The location of offsets shifted more upfield as the distance between the methyl group and the polar group became further. Please see FIG. 4A. The magnitude of CEST signal also went up as the number of methyl groups increased. There was also a broad dip in the 0.5 ppm to 1.5 ppm range in Z-spectra of alcohols, which might be related to the exchangeable hydroxyl group in their structures. MTR_{asym} of alcohols also showed the presence of a peak around 1 ppm (please see FIGS. 8A to 8H), while in aprotic analogs such peak was not present. Please see FIGS. 7A to 7F.

### Physicochemical Properties of BA-DMSO Liposome

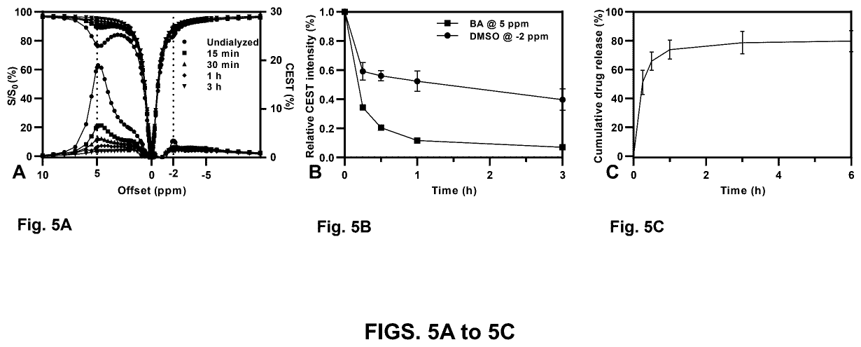

Freshly prepared BA-DMSO liposome had a size of 161.03 nm, PDI of 0.07, and Zeta-potential of −1.47 mV, concentration of (1.45±0.37)×10⁸ particles per mL (n=3). They also showed two obvious peaks at 5 ppm and −2 ppm, originating from encapsulated BA and DMSO respectively. Dialysis results showed that CEST contrast decreased by more than half in 15 minutes, and contrast became less distinguishable at 1 hour (please see FIG. 5A). The retention of BA in BA-DMSO liposome was calculated to be 55 mM before dialysis, and 5.87 mM after dialysis. From the release curve, about 80% of BA content were released in 6 hours (please see FIG. 5C).

### Discussions and Conclusions

This present invention has demonstrated that CEST effects of commonly used solvent systems for drug delivery, including aprotic and protic solvents. It is the first report of the CEST effects of common solvents at negative offset frequencies, where the conventionally known rNOE signals are also resonating at, from the structure of DMSO. This unique CEST effect exhibited rNOE-like characteristics, such as less sensitivity to pH and temperature, suggesting the contrast mechanism behind DMSO's CEST signal could share similarities with rNOE signals. In rNOE signals, exchangeable hydroxyl group on the mobile macromolecule, immobile semisolid, and immobile receptor can serve as a relaying site for magnetization transfer from RF-labeled aliphatic proton. However, such macromolecules or immobilized substrates are not present in the DMSO-water mixture. Based on spectroscopic studies of DMSO's interaction with water, the intermolecular interaction between DMSO and water creates a solvation shell, where water molecules in the solvation shell are more structured, rigid, and close to DMSO molecules. With this in mind, it appears that this “structured” water serves as relaying sites, where RF-labeled aliphatic protons of methyl groups of DMSO can transfer magnetization to. Subsequently, proton exchange can take place between “structured” water and bulk water, resulting in the observable peak we observed at −2 ppm (please see FIG. 1A).

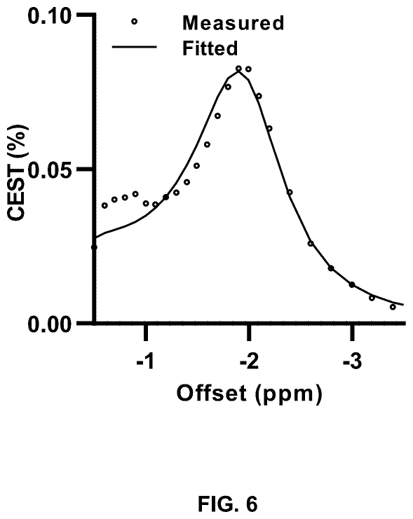

During the course leading to the present invention, a series of internal and external factors that can influence DMSO's CEST effect were examined. DMSO's concentration linearly correlated with observed signal magnitude, while the change in signal when varying pH from pH 5.5 to pH 8 was less obvious. As temperature went down from 40° C. to 20° C., DMSO's CEST peak gradually moved upfield (please see FIGS. 1F to 1G). It appears that intermolecular interaction between DMSO and water was dependent on temperature. For external factors, DMSO's CEST signal slightly increased when power went up from 0.4 μT to 2 μT, and for varying Tₛₐₜ the signal magnitude was relatively stable. Exchange rate and was estimated with Bloch-McConnell fitting. Input of solute concentration was 1.41 M (molar concentration of 10% v/v DMSO) multiplied by a factor of 1.4, which was an estimation of concentration of “structured” water that formed by intermolecular interaction with DMSO and thereby served as relaying sites. From the fitting result (please see FIG. 6), the estimated solute concentration was 1.96 M, which was very close to the input concentration of 1.97 M. The estimated exchange rate was around 2 Hz (please see below Table 2), which falls into a very slow exchange regime.

TABLE 2
Parameters used in Bloch-McConell fitting and the fitted
value for exchanging proton fraction, exchange rate,
longitudinal and transverse relaxation rate.
Fitting parameter

| B₀ | 3T | |
|---|---|---|
| B₁ | 1.2 | μT |
| Tₛₐₜ | 4 | s |
| Peak range | −0.5 to −2.5 | ppm |
| Water Molarity | 55.5M | |
| Water Proton concentration | 111M | |
| Water T₁ₐ | 3.633 | s |
| Water T₂ₐ | 1.887 | s |
| DMSO Molarity | 1.41M | |
| DMSO coupled proton concentration | 1.41*1.4M | |
| DMSO coupled proton fraction | 0.0178 | |
| DMSO T_{1b} | 3.00 | s |

Fitted values

| B₁ inhomogeneity | 0.8697 | |
|---|---|---|
| Kₑₓ | 2.1341 | s⁻¹ |
| DMSO T_{2b} | 0.4607 | s |

In further study of structural analogs of DMSO, acetone and acetonitrile are both polar aprotic solvents that are known to have intermolecular interaction with neighboring water, in a similar way as DMSO does. They also showed CEST peaks around −2.5 ppm, and about 1-2% less in signal magnitude compared with DMSO of the same concentration (please see FIG. 3). Four members from the alcohol family also had distinctive CEST peaks, providing more insight that offset and signal magnitude could be affected by the distance between —CH and the polar group (please see FIG. 4). For DMSO, acetone, acetonitrile, and tert-butyl-alcohol, regional reordering of water were observed in spectroscopy and molecular dynamic studies, indicating that the solvation shell of “structured” water plays a central role in generating the observed CEST contrast for both protic and aprotic solvents.

Liposome is a widely used nanocarrier for anti-cancer, anti-fungal, and analgesic drugs, as well as vaccine, and DMSO solvent system has been applied to increase the payload for liposomal anticancer drugs. Liposome formulation can be fine-tuned to achieve higher payload, controlled release, and adjustable targeting properties by adjusting the composition of the phospholipid bilayer, e.g., polyethylene glycol and cholesterol content. Additionally, endogenous and exogeneous diamagnetic compounds with exchangeable protons, including L-arginine, barbituric acid, and citicoline, can be encapsulated into liposome and achieved in vivo monitoring of the compound with CEST MRI. The liposomal CEST contrast can be further optimized through controlling the size of liposomes. DMSO now holds the potential of a CEST-trackable drug delivery excipient, especially for those hydrophobic drugs. To demonstrate the feasibility, we dissolved BA, which has a CEST peak at 5 ppm, in 10% DMSO, and incorporated them in liposomes. From FIG. 5A to 5C, two distinctive peaks are observed in the liposome solution, and with rapid decrease in contrast within an hour of dialysis against HBS. This is attributed to the concentration gradient inside and outside dialysis membrane. At the end of dialysis, around 80% of the BA content was released.

In the present invention, we have demonstrated for the first time that the common solvent DMSO had a CEST peak at around −2 ppm and the DMOS analogs have a CEST peak in the range −1.4 ppm to −3.8 ppm. CEST properties of DMSO and its structural analogs were measured, and the present invention shows that the DMSO CEST effect originates from the solvation shell formed between the solvent molecules and water. The present invention also shows the release of BA-DMSO co-loaded in liposome can be monitored at their respective CEST peaks.

It should be understood that certain features of the invention, which are, for clarity, described in the content of separate embodiments, may be provided in combination in a single embodiment. Conversely, various features of the invention which are, for brevity, described in the content of a single embodiment, may be provided separately or in any appropriate sub-combinations. It is to be noted that certain features of the embodiments are illustrated by way of non-limiting examples.

### REFERENCES

The following references are incorporated in their entirety and a skilled person is considered to be aware of disclosure of these references.
Wu X F, Natte K. The Applications of Dimethyl Sulfoxide as Reagent in Organic Synthesis. Adv Synth Catal. Feb. 4, 2016; 358(3):336-352. doi:10.1002/adsc.201501007
Jang T H, Park S C, Yang J H, et al. Cryopreservation and its clinical applications. Integr Med Res. March 2017; 6(1):12-18. doi:10.1016/j.imr.2016.12.001
Capriotti K, Capriotti J A. Dimethyl sulfoxide: history, chemistry, and clinical utility in dermatology. J Clin Aesthet Dermatol. September 2012; 5(9):24-6.
Yeh M-K, Chang L-C, Chiou A H-J. Improving Tenoxicam Solubility and Bioavailability by Cosolvent System. AAPS PharmSciTech. 2009; 10(1):166-171. doi:10.1208/s12249-009-9189-2
Jeliński T, Stasiak D, Kosmalski T, Cysewski P. Experimental and Theoretical Study on Theobromine Solubility Enhancement in Binary Aqueous Solutions and Ternary Designed Solvents. Pharmaceutics. 2021; 13(8):1118. doi:10.3390/pharmaceutics13081118
Shakeel F, Alshehri S, Imran M, Haq N, Alanazi A, Anwer M K. Experimental and Computational Approaches for Solubility Measurement of Pyridazinone Derivative in Binary (DMSO+Water) Systems. Molecules. 2019; 25(1):171. doi:10.3390/molecules25010171
Mizuno K, Imafuji S, Ochi T, Ohta T, Maeda S. Hydration of the CH groups in dimethyl sulfoxide probed by NMR and IR. J Phys Chem B. Nov. 30, 2000; 104(47):11001-11005. doi:DOI 10.1021/jp001079x
Vaisman I I, Berkowitz M L. Local Structural Order and Molecular Associations in Water Dmso Mixtures—Molecular-Dynamics Study. J Am Chem Soc. Sep. 23, 1992; 114(20):7889-7896. doi:DOI 10.1021/ja00046a038
Bertoluzza A, Bonora S, Battaglia M A, Monti P. Raman and Infrared Study on the Effects of Dimethylsulfoxide (Dmso) on Water-Structure. J Raman Spectrosc. 1979; 8(5):231-235. doi:DOI 10.1002/jrs.1250080502
Roy S, Banerjee S, Biyani N, Jana B, Bagchi B. Theoretical and Computational Analysis of Static and Dynamic Anomalies in Water—DMSO Binary Mixture at Low DMSO Concentrations. J Phys Chem B. Feb. 3, 2011; 115(4):685-692. doi:10.1021/jp109622h
Balakin K V, Savchuk N P, Tetko I V. In silico approaches to prediction of aqueous and DMSO solubility of drug-like compounds: trends, problems and solutions. Curr Med Chem. 2006; 13(2):223-41. doi:10.2174/092986706775197917
van Zijl P C M, Yadav N N. Chemical Exchange Saturation Transfer (CEST): What is in a Name and What Isn't? Magn Reson Med. April 2011; 65(4):927-948. doi:10.1002/mrm.22761
Chen L Q, Howison C M, Jeffery J J, Robey I F, Kuo P H, Pagel M D. Evaluations of extracellular pH within in vivo tumors using acidoCEST MRI. Magn Reson Med. 2014; 72(5):1408-1417. doi:10.1002/mrm.25053
Liu G S, Qin Q, Chan K W Y, et al. Non-invasive temperature mapping using temperature-responsive water saturation shift referencing (T-WASSR) MRI. Nmr Biomed. March 2014; 27(3):320-331. doi:10.1002/nbm.3066
Yang X, Song X L, Li Y G, et al. Salicylic Acid and Analogues as diaCEST MRI Contrast Agents with Highly Shifted Exchangeable Proton Frequencies. Angew Chem Int Edit. Jul. 29, 2013; 52(31):8116-8119. doi:10.1002/anie.201302764
Yuan Y, Zhang J, Qi X L, et al. Furin-mediated intracellular self-assembly of olsalazine nanoparticles for enhanced magnetic resonance imaging and tumour therapy. Nat Mater. December 2019; 18(12):1376-+. doi:10.1038/s41563-019-0503-4
Zhou J Y, Payen J F, Wilson D A, Traystman R J, van Zijl P C M. Using the amide proton signals of intracellular proteins and peptides to detect pH effects in MRI. Nat Med. August 2003; 9(8):1085-1090. doi:10.1038/nm907
Zhou J Y, Lal B, Wilson D A, Laterra J, van Zijl P C M. Amide proton transfer (APT) contrast for imaging of brain tumors. Magn Reson Med. December 2003; 50(6):1120-1126. doi:10.1002/mrm.10651
Kogan F, Haris M, Singh A, et al. Method for High-Resolution Imaging of Creatine In Vivo Using Chemical Exchange Saturation Transfer. Magn Reson Med. January 2014; 71(1):164-172. doi:10.1002/mrm.24641
Haris M, Nanga R P R, Singh A, et al. Exchange rates of creatine kinase metabolites: feasibility of imaging creatine by chemical exchange saturation transfer MRI. NMR Biomed. 2012; 25(11):1305-1309.
Cai K J, Haris M, Singh A, et al. Magnetic resonance imaging of glutamate. Nat Med. February 2012; 18(2):302-306. doi:DOI 10.1038/nm.2615
Davis K A, Nanga R P R, Das S, et al. Glutamate imaging (GluCEST) lateralizes epileptic foci in nonlesional temporal lobe epilepsy. Science translational medicine. 2015; 7(309):309ra161-309ra161.
Chan K W Y, McMahon M T, Kato Y, et al. Natural D-glucose as a biodegradable MRI contrast agent for detecting cancer. Magn Reson Med. November 2012; 68(6):1764-1773. doi:10.1002/mrm.24520
Walker-Samuel S, Ramasawmy R, Torrealdea F, et al. In vivo imaging of glucose uptake and metabolism in tumors. Nat Med. 2013; 19(8):1067-1072. doi:10.1038/nm.3252
Huang J P, van Zijl P C M, Han X Q, et al. Altered D-glucose in brain parenchyma and cerebrospinal fluid of early Alzheimer's disease detected by dynamic glucose-enhanced MRI. Sci Adv. May 2020; 6(20)doi:ARTN eaba3884 10.1126/sciadv.aba3884
van Zijl P C M, Lam W W, Xu J D, Knutsson L, Stanisz G J. Magnetization Transfer Contrast and Chemical Exchange Saturation Transfer MRI. Features and analysis of the field-dependent saturation spectrum. Neuroimage. March 2018; 168:222-241. doi:10.1016/j.neuroimage.2017.04.045
Xu X, Yadav N N, Zeng H F, et al. Magnetization Transfer Contrast-Suppressed Imaging of Amide Proton Transfer and Relayed Nuclear Overhauser Enhancement Chemical Exchange Saturation Transfer Effects in the Human Brain at 7T. Magn Reson Med. January 2016; 75(1):88-96. doi:10.1002/mrm.25990
Jones C K, Huang A, Xu J D, et al. Nuclear Overhauser enhancement (NOE) imaging in the human brain at 7 T. Neuroimage. Aug. 15, 2013; 77:114-124. doi:10.1016/j.neuroimage.2013.03.047
Goerke S, Soehngen Y, Deshmane A, et al. Relaxation-compensated APT and rNOE CEST-MRI of human brain tumors at 3 T. Magn Reson Med. August 2019; 82(2):622-632. doi:10.1002/mrm.27751
Huang J P, Xu J D, Lai J H C, et al. Relayed nuclear Overhauser effect weighted (rNOEw) imaging identifies multiple sclerosis. Neuroimage-Clin. 2021; 32doi:ARTN 102867 10.1016/j.nicl.2021.102867
Huang J P, Lai J H C, Tse K H, et al. Deep neural network based CEST and AREX processing: Application in imaging a model of Alzheimer's disease at 3 T. Magn Reson Med. March 2022; 87(3):1529-1545. doi:10.1002/mrm.29044
Zhou Y, van Zijl P C M, Xu X, et al. Magnetic resonance imaging of glycogen using its magnetic coupling with water. P Natl Acad Sci USA. Feb. 11, 2020; 117(6):3144-3149. doi:10.1073/pnas.1909921117
Zu Z L. Ratiometric NOE(-1.6) contrast in brain tumors. Nmr Biomed. December 2018; 31(12)doi:ARTN e4017 10.1002/nbm.4017
Huang J P, Han X Q, Chen L, Xu X, Xu J D, Chan K W Y. Relayed nuclear Overhauser enhancement imaging with magnetization transfer contrast suppression at 3 T. Magn Reson Med. January 2021; 85(1):268-281. doi:10.1002/mrm.28433
Yadav N N, Yang X, Li Y G, Li W B, Liu G S, van Zijl P C M. Detection of dynamic substrate binding using MRI. Sci Rep-Uk. Aug. 31, 2017; 7doi:ARTN 10138 10.1038/s41598-017-10545-1
Filipczak N, Pan J Y, Yalamarty S S K, Torchilin V P. Recent advancements in liposome technology. Adv Drug Deliver Rev. 2020; 156:4-22. doi:10.1016/j.addr.2020.06.022
Barenholz Y. Doxil®—The first FDA-approved nano-drug: Lessons learned. J Control Release. Jun. 10, 2012; 160(2):117-134. doi:10.1016/j.jconrel.2012.03.020
Han Z, Liu G S. CEST MRI trackable nanoparticle drug delivery systems. Biomed Mater. Mar. 1, 2021; 16(2)doi:ARTN 024103 10.1088/1748-605X/abdd70
Ferrauto G, Terreno E. Compartmentalized agents: A powerful strategy for enhancing the detection sensitivity of chemical exchange saturation transfer contrast. Nmr Biomed. June 2023; 36(6)doi:ARTN e4791 10.1002/nbm.4791
Zhao J M, Har-El Y E, McMahon M T, et al. Size-induced enhancement of chemical exchange saturation transfer (CEST) contrast in liposomes. J Am Chem Soc. Apr. 16, 2008; 130(15):5178-5184. doi:10.1021/ja710159q
Han X Q, Huang J P, To A K W, et al. CEST MRI detectable liposomal hydrogels for multiparametric monitoring in the brain at 3T. Theranostics. 2020; 10(5):2215-2228. doi:10.7150/thno.40146
Law L H, Huang J P, Xiao P, et al. Multiple CEST contrast imaging of nose-to-brain drug delivery using iohexol liposomes at 3T MRI. J Control Release. February 2023; 354:208-220. doi:10.1016/j.jconrel.2023.01.011
Huang J P, Chen Z L, Park S W, Lai J H C, Chan K W Y. Molecular Imaging of Brain Tumors and Drug Delivery Using CEST MRI: Promises and Challenges. Pharmaceutics. February 2022; 14(2)doi:ARTN 451 10.3390/pharmaceutics14020451
Zaiss M, Schmitt B, Bachert P. Quantitative separation of CEST effect from magnetization transfer and spillover effects by Lorentzian-line-fit analysis of z-spectra. J Magn Reson. 2011; 211(2):149-155.
Zhou Y, Bie C X, van Zijl P C M, Yadav N N. The relayed nuclear Overhauser effect in magnetization transfer and chemical exchange saturation transfer MRI. Nmr Biomed. June 2023; 36(6)doi:ARTN e4778 10.1002/nbm.4778
Zhou Y, Bie C X, van Zijl P C M, Xu J D, Zou C, Yadav N N. Detection of electrostatic molecular binding using the water proton signal. Magn Reson Med. August 2022; 88(2):901-915. doi:10.1002/mrm.29230
Mancera R L, Chalaris M, Refson K, Samios J. Molecular dynamics simulation of dilute aqueous DMSO solutions. A temperature-dependence study of the hydrophobic and hydrophilic behaviour around DMSO. Phys Chem Chem Phys. 2004; 6(1):94-102. doi:10.1039/b308989d
Zaiss M, Schnurr M, Bachert P. Analytical solution for the depolarization of hyperpolarized nuclei by chemical exchange saturation transfer between free and encapsulated xenon (HyperCEST). J Chem Phys. Apr. 14, 2012; 136(14)doi:Artn 144106 10.1063/1.3701178
Zaiss M, Bachert P. Exchange-dependent relaxation in the rotating frame for slow and intermediate exchange-modeling off-resonant spin-lock and chemical exchange saturation transfer. Nmr Biomed. May 2013; 26(5):507-518. doi:10.1002/nbm.2887
Oh K I, Rajesh K, Stanton J F, Baiz C R. Quantifying Hydrogen-Bond Populations in Dimethyl Sulfoxide/Water Mixtures. Angew Chem Int Edit. Sep. 11, 2017; 56(38):11375-11379. doi:10.1002/anie.201704162
Large D E, Abdelmessih R G, Fink E A, Auguste D T. Liposome composition in drug delivery design, synthesis, characterization, and clinical application. Adv Drug Deliver Rev. September 2021; 176doi:ARTN 113851 10.1016/j.addr.2021.113851
Ahad A, Shakeel F, Raish M, et al. Thermodynamic Solubility Profile of Temozolomide in Different Commonly Used Pharmaceutical Solvents. Molecules. February 2022; 27(4)doi:ARTN 1437 10.3390/molecules27041437
Mickey D D, Carvalho L, Foulkes K. Conventional chemotherapeutic agents combined with DMSO or DFMO in treatment of rat prostate carcinoma. Prostate. 1989; 15(3):221-32. doi:10.1002/pros.2990150303
Chan K W Y, Bulte J W M, McMahon M T. Diamagnetic chemical exchange saturation transfer (diaCEST) liposomes: physicochemical properties and imaging applications. Wires Nanomed Nanobi. January 2014; 6(1):111-124. doi:10.1002/wnan.1246
Liu G S, Moake M, Har-el Y E, et al. In Vivo Multicolor Molecular MR Imaging Using Diamagnetic Chemical Exchange Saturation Transfer Liposomes. Magn Reson Med. April 2012; 67(4):1106-1113. doi:10.1002/mrm.23100
Chan K W Y, Liu G S, Song X L, et al. MRI-detectable pH nanosensors incorporated into hydrogels for in vivo sensing of transplanted-cell viability. Nat Mater. March 2013; 12(3):268-275. doi:10.1038/Nmat3525
Chan K W Y, Yu T, Qiao Y, et al. A diaCEST MRI approach for monitoring liposomal accumulation in tumors. J Control Release. Apr. 28, 2014; 180:51-59. doi:10.1016/j.jconrel.2014.02.005
Liu H L, Jablonska A, Li Y G, et al. Label-free CEST MRI Detection of Citicoline-Liposome Drug Delivery in Ischemic Stroke. Theranostics. 2016; 6(10):1588-1600. doi:10.7150/thno.15492
Ward K M, Aletras A H, Balaban R S. A new class of contrast agents for MRI based on proton chemical exchange dependent saturation transfer (CEST). J Magn Reson. March 2000; 143(1):79-87. doi:DOI 10.1006/jmre.1999.1956
Lipinski, C. (2008). Drug solubility in water and dimethylsulfoxide. Molecular drug properties, 255-282.
Hoang, C., Nguyen, A. K., Nguyen, T. Q., Fang, W., Han, B., Hoang, B. X., & Tran, H. D. (2021). Application of dimethyl sulfoxide as a therapeutic agent and drug vehicle for eye diseases. Journal of Ocular Pharmacology and Therapeutics, 37(8), 441-451.
Karim, M., Boikess, R. S., Schwartz, R. A., & Cohen, P. J. (2023). Dimethyl sulfoxide (DMSO): a solvent that may solve selected cutaneous clinical challenges. Archives of Dermatological Research, 315(6), 1465-1472.
Terwogt, J. M., Nuijen, B., Huinink, W. T. B., & Beijnen, J. H. (1997). Alternative formulations of paclitaxel. Cancer treatment reviews, 23(2), 87-95.
Tinjacá, D. A., Martínez, F., Almanza, O. A., Peña, M., Jouyban, A., & Acree, W. E. (2022). Increasing the equilibrium solubility of meloxicam in aqueous media by using dimethyl sulfoxide as a cosolvent: Correlation, dissolution thermodynamics and preferential solvation. Liquids, 2(3), 161-182.
Ko, H. H., Lareu, R. R., Dix, B. R., & Hughes, J. D. (2018). In vitro antibacterial effects of statins against bacterial pathogens causing skin infections. European Journal of Clinical Microbiology & Infectious Diseases, 37, 1125-1135.
Chen, X., Zhang, Y., Liu, C., Zhang, Y., Zhou, X., Zhou, T., . . . & Li, J. (2010). Retinoic acid and dimethyl sulfoxide promote efficient delivery of transgenes to mouse skin by topically transdermal penetration. Drug Delivery, 17(6), 385-390.
Heckert, R. A., Elankumaran, S., Oshop, G. L., & Vakharia, V. N. (2002). A novel transcutaneous plasmid-dimethylsulfoxide delivery technique for avian nucleic acid immunization. Veterinary immunology and immunopathology, 89(1-2), 67-81.
Oshop, G. L., Elankumaran, S., Vakharia, V. N., & Heckert, R. A. (2003). In ovo delivery of DNA to the avian embryo. Vaccine, 21(11-12), 1275-1281.

What is claimed is:

1. A system for delivery of a drug in vivo wherein the extent of the delivery of the drug is monitorable and/or controllable by using chemical exchange saturation transfer magnetic resonance imaging (CEST MRI), comprising a solvent system of dimethyl sulfoxide (DMSO) or its analogs for carrying the drug, detecting the delivery and/or biodistribution the drug, and/or interaction of the drug compartmentalized in vivo by way of directly imaging the solvent system by the CEST MRI due to the inherent contrast of the solvent system.

2. A system as claimed in claim 1, wherein the drug is a hydrophobic drug, an anticancer drug, a small molecule drug, or a macromolecular drug selected from a group including a DNA, RNA, or peptide.

3. A system as claimed in claim 1, wherein the drug is free of any label.

4. A system as claimed in claim 1, wherein the solvent system or the drug carried by the solvent system is free of any metallic, radioactive or fluorescent dye or label.

5. A system as claimed in claim 2, wherein when the drug is a hydrophobic drug the concentration of the hydrophobic drug in the solvent system is at 1 mM to 45 mM, or when the drug is a macromolecular drug the concentration of the macromolecular drug is at 0.1 μg/μL to 2 μg/μL.

6. A system as claimed in claim 1, wherein the concentration of the DMSO or its analogs in the solvent is 1.25 to 20% v/v.

7. A system as claimed in claim 1, wherein CEST effects of the DMSO or its analogs range from −1.4 ppm to −3.8 ppm.

8. A system as claimed in claim 1, wherein the analogs of the solvent system are selected from a group including aprotic solvents and protic solvents.

9. A system as claimed in claim 8, wherein the analogs of the solvent system are selected from the group consisting of acetone, acetonitrile, methanol, ethanol, 1-propanol and 2-propanol.

10. A system as claimed in claim 8, wherein the aprotic and protic solvents of the analogs are both polar and having methyl groups therein.

11. A system as claimed in claim 1, comprising a nanoparticle carrier for the drug, wherein the nanoparticle carrier is liposome.

12. A system as claimed in claim 11, wherein the solvent system loaded with drug is then co-loaded with the liposome.

13. A system as claimed in claim 11, wherein the liposome is used as a probe for monitoring of delivery of the drug.

14. A system as claimed in claim 1, further comprising a MRI-trackable nanocarrier of the drug in the solvent system.

15. A system as claimed in claim 14, wherein the MRI-trackable nanocarrier is adapted to be controllable for release of the drug when the drug reaches certain location or circumstances in vivo.

16. A method of using chemical exchange saturation transfer magnetic resonance imaging (CEST MRI) for monitoring delivery and/or controlling a release of a drug in vivo, comprising the steps of:
providing a drug to be delivered,
providing a solvent system of dimethyl sulfoxide (DMSO) or its analogs for carrying the drug,
using CEST MRI to detect the delivery and/or biodistribution of the drug, and/or interaction of the drug compartmentalized in vivo by way of directly imaging the solvent system by the CEST MRI due to the inherent contrast of the solvent system.

17. A method as claimed in claim 16, wherein the drug is selected from a group selected from a hydrophobic drug, an anticancer drug, a small molecule drug, a macromolecular drug, including peptide, DNA and RNA, and the drug is free of any label, and/or the solvent or the drug is free of any metallic, radioactive or fluorescent dye or label.

18. A method as claimed in claim 16, wherein when the drug is a hydrophobic drug the concentration of the hydrophobic drug in the solvent system is at 1 mM to 45 mM, or when the drug is a macromolecular drug the concentration of the macromolecular drug is at 0.1 μg/μL to 2 μg/μL, and the concentration of the DMSO or its analogs in the solvent is 1.25 to 20% v/v.

19. A method as claimed in claim 16, wherein the solvent of analogs is selected from the group including aprotic solvents and protic solvents, or is acetone, acetonitrile, methanol, ethanol, 1-propanol or 2-propanol.

20. A method as claimed in claim 1, comprising a nanoparticle carrier for the drug, wherein the nanoparticle carrier is liposome, and wherein the solvent system loaded with the drug is then co-loaded with the liposome, wherein the liposome is used as a probe for monitoring of delivery of the drug.

21. A method as claimed in claim 1, further comprising a MRI-trackable nanocarrier of the drug in the solvent system, wherein the MRI-trackable nanocarrier is adapted to be controllable for release of the drug when the drug reaches certain location or circumstances in vivo.
